(19) United States
(12) Patent Application Publication
(10) Pub. No.: US 2007/0190591 A1
(43) Pub. Date: Aug. 16, 2007

(54) Microbial compositions and methods of use in water

(76) Inventors: Nigel M. Grech, Reedley, CA (US); Shinn Liang Lin, San Gabriel, CA (US)

Correspondence Address: PERKINS COIE LLP, POST OFFICE BOX 1208, SEATTLE, WA, 98111-1208, US

(21) Appl. No.: 11/516,241

(22) Filed: Sep. 5, 2006

Related U.S. Application Data
(63) Continuation-in-part of application No. 10/644,583, filed on Aug. 19, 2003, now abandoned.
Application No. 10/644,583 is a continuation of application No. 09/848,102, filed on May 2, 2001, now Pat. No. 6,632,363.

(51) Int. Cl. C12Q 1/02

(52) U.S. Cl. 435/29

(57) ABSTRACT

Methods and compositions using an isolate Bacillus subtilis strain for treating a sample are disclosed. The methods and compositions disclosed are used in samples to affect a change in the sample such as cleaning the samples of unwanted elements or organisms (e.g., the presence or overgrowth of algae or fungi).

### CROSS-REFERENCE TO RELATED APPLICATIONS

This application is a continuation-in-part of U.S. patent application Ser. No. 10/644,583, filed Aug. 19, 2003, which is a continuation of U.S. patent application Ser. No. 09/848,102, filed May 2, 2001, now U.S. Pat. No. 6,632,363, the disclosures of which are incorporated herein by reference in their entirety.

### FIELD OF THE INVENTION

The present invention relates to the field of compositions comprising an isolated strain of Bacillus subtilis strain and methods of use thereof.

### BACKGROUND OF THE INVENTION

Microbes and microbial preparations are becoming increasingly important and effective in combating pests and diseases.

Currently, the following methods are employed to control algae blooms and other unwanted plants and organisms. One method involves the use of chemical poisons. For example, the most commonly used algaecides include copper sulfate and diuron which have detrimental environmental impact, are regulated by the EPA as well as state agencies, require expert handling, and are considered pollutants. Application often requires the use of a boat or other device to deliver the pesticide to the target organism as persons must spray the treatment directly onto the target organism or plant. Application is also very labor intensive, time consuming, and a danger to persons applying the product. These and other pesticides, herbicides, and pollutants for the control of algae or other noxious plants and organisms are constantly under heavy scrutiny by municipal, state, and federal regulators.

Another current method involves the use of colorings and shadings that provide limited control of the targeted organism or plant through prevention (blocking sunlight that is an essential stimulant for many algae that thrive on photosynthesis). Consequently, beneficial and ornamental water plants requiring sunlight are also deprived of needed light and do not thrive. Additionally, water clarity is sacrificed and unnaturally colored water is created by the dyes, which is undesirable in many circumstances.

Another method involves the application of ultra-violet light in the form of a pre-filter to the area water gardening and koi ponds. Ultra-violet filtration, as it is commonly called, kills unwanted as well as beneficial bacteria. The result is a pond more reliant on chemical fixes since natural pond balancing, nitrogen fixing, and micro-flora are destroyed. Additionally, ultra-violet filtration can not be practically applied to larger bodies of water or naturally sustaining bodies of water.

Another current method involves microbial inoculation of water, which has generally taken two forms, that of a liquid solution of microbes and a granular blend. Neither form, however, offers an effective distribution method. Extensive mechanical agitation is required in order to distribute a liquid microbial inoculate throughout the water. Without extensive mechanical agitation, these liquid microbial formulas tend to remain concentrated at the origin of application. Additionally, liquid microbial formulas with viscosities that are greater or less than water will tend to hover or sink in a specific location.

The other form of microbial inoculate currently available is granular. These granular formulas are typically similar in weight to that of beach sand. When introduced into the treatment waters, these granular formulations fall directly to the bottom. Only a very tiny percentage of the granular formulation (typically in the form of wheat germ) is left on the surface immediately following treatment. This method of granular microbial inoculation is largely ineffective, as the beneficial microbes are not effectively nor efficiently delivered to the target organism (alga, fungi, weeds, diseased fish, etc).

Each of the methods described above are deficient in their effectiveness since the composition applied to the algae, noxious weed, or target organism is concentrated at the origin of application and the poisons, antibiotics, or microbes are not evenly distributed. Further, since the application of the composition is limited to the localized area of application, only those algae or other plants or organisms residing in that localized area of the body of water will be treated sufficiently with the correct dosage. The target organisms residing in the adjacent water columns will not be treated as effectively. Thus, the current methods are inefficient for the treatment of algae, plants, or fungi in water applications.

Thus, composition and methods for treating algae, fungi, noxious weeds, or unwanted plants in a sample are desirable. For example, compositions and methods for delivering microbes to water uniformly and efficiently over the water surface and down through the water for the treatment of pests or diseases would offer substantial benefits over the current techniques. Accordingly, objects of this invention compositions for use in a sample, wherein the composition distributes microbes uniformly and efficiently throughout the sample. Another object of the present invention is a method for applying microbes uniformly and efficiently over a water surface and down through the water. Another object of the present invention is to deliver microbes proportionately to the target alga, weeds, or organisms by blanketing the target pest effectively with the microbe.

### SUMMARY OF THE INVENTION

One aspect of the present invention relates to compositions for controlling, treating, regulating, modulating, alleviating, reducing, preventing, or cleansing a condition of a sample. In one embodiment, the composition comprises a microbe agent which is an isolated, substantially biologically pure strain of Bacillus subtilis which was deposited under American Type Culture Collection with a patent deposit designation number PTA-7736 (referred herein as “ATCC PTA-7736”).

One embodiment of the present invention is to provide a composition and method by which the compositions can be used to treat or clean a water application. In another embodiment, the microbe agent is distributed over a water surface and released into the body of water by using the compositions which further comprise hydrophobic carriers. In a preferred embodiment, the distribution can be made uniformly and efficiently

Another aspect of the present invention is directed to a hydrophobic composition used for the delivery of a microbial agent to water comprising a hydrophobic carrier and a microbial agent.

Another aspect of the present invention relates to the method for controlling, treating, regulating, modulating, alleviating, reducing, preventing; or cleansing a condition of a sample by delivering the compositions to a sample. In one embodiment, the composition is delivered uniformly and/or efficiently.

Another aspect of the invention relates to a method of administering a microbial agent to water, preferably uniformly and efficiently, by introducing a hydrophobic composition containing a microbe agent to the water application and delivering it to a target plant or organism.

Another aspect of the invention is a method for improving the water quality in water by introducing hydrophobic compositions to the water.

### BRIEF DESCRIPTION OF THE DRAWINGS

FIG. 1: Comparison of untreated (left) vs. B. subtilis treated (right) Algal cultures.

FIG. 2: Before (left) and after (right) application of B. subtilis in algae infested lake.

FIG. 3: B. subtilis antagonism towards a plant pathogenic fungus, Fusarium oxysporum.

### DETAILED DESCRIPTION

One aspect of the present invention relates to compositions for controlling, treating, regulating, modulating, alleviating, reducing, preventing, or cleansing a condition of a sample.

As used herein, a composition comprises a microbial agent, which is an isolated, substantially biologically pure Bacillus subtilis strain. The strain was deposited under American Type Culture Collection with a patent deposit designation number of PTA-7736, referred herein as ATCC PTA-7736. The ATCC PTA-7736 strain was isolated from a mixture of Bacillus subtilis strains and discovered unexpectedly to perform unexpected success in treating or cleansing a sample. The composition can further comprise a hydrophilic carrier, a liquid carrier, a solid carrier, a hydrophobic carrier, or a pharmaceutically acceptable carrier. The composition can further comprise bacteria, nematodes, protozoan or combinations thereof.

A condition used herein refers to the presence or overgrowth of unwanted organisms such as fungi, algae, and weeds in, on, or surrounding a sample and the results thereof (e.g., a disease). Thus, controlling, alleviating, reducing, preventing, suppressing, eliminating, eradicating, or cleansing a condition becomes desirable. It is further preferable to prevent, control, alleviate, or treat a disease resulting from the presence or overgrowth of unwanted organisms. For example, as disclosed herein, a condition can be fungi related diseases of human and animals, wherein animal refers to any member of the animal kingdom that is not a human being; a condition can be mould in residential or commercial buildings; a condition can also be overgrowing of algae in water.

A sample used herein refers to any subject that has a propensity to have the presence or overgrowth of unwanted organisms (e.g., algae and/or fungi) in, on, surrounding, or in mix with the subject, the consequence of which may lead to a condition. Examples of the sample include, for instance, liquid (e.g., water), gas (e.g., air), solid matter (e.g., soil), a life form (e.g., a plant, a human, or an animal), and a tangible article (e.g., a building).

In one embodiment of the present invention, liquid include water and any matter in the phase of fluid. As used herein, water includes bodies of water such as lakes, rivers, canals, ponds, and water holding structures such as tanks and reservoirs. Also contemplated are natural and man made irrigation systems and water films on the surface of plants. It should be clear to anyone reading this disclosure that water in any form is contemplated as a possible sample for the compositions and methods disclosed herein. A condition of water refers to, for example, overgrowth of algae or fungi in the water.

In another embodiment of the present invention, gas is air. Examples of air include, but are not limited to, the atmosphere, the processed air, the air confined in a close container or space (e.g., the air in a building or a car). The condition of air refers to, for example, the presence or overgrowth of fungi or mold in the air, in which case a composition comprising the ATCC PTA-7736 strain (or the strain with any other carriers) can be sprayed to the air or applied to an air condition filter to treat, cleanse, or prevent the presence or overgrowth of fungi or mold in the air.

In another embodiment of the present invention, a solid matter is soil or any matter in its solid phase. Soil is the complex body on the surface of a lithosphere subject to soil formation processes, comprising mineral and organic matter, as well as living organisms. Also contemplated is soil that is relocated from its original location to a container or any other position. A condition of soil includes, but are not limited to, the presence or overgrowth of fungi and/or algae in the soil, which becomes desirable to control, alleviate, eradicate, prevent, cleanse the said condition.

The algae in soil include, but are not limited to, Microscopic, Filimentous and blue-green algae. The fungi in soil include, but are not limited to, Phytophthora, Pythium, Verticillium, Rhizoctonia, and Fusarium. The overgrowth of algae and/or fungi in soil may lead to soil-related plant diseases which include, for example, Phytophthora, Pythium, Verticillium, Rhizoctonia, and Fusarium. The composition of the present invention comprising the ATCC PTA-7736 strain (or the strain in combination with other carriers) can be applied to soil by spraying, watering, irrigation application, or directly mixing or adding. For example, the composition can be added into soil which is then placed in containers where plants grow to prevent or treat soil-related plant diseases resulting from overgrowth of algae or fungi in soil.

In another embodiment of the present invention, a life form is a plant. Examples of plants include trees, flowers, leaves, trunks, branches, fruits, roots, vegetables, herbs, ferns, and mosses. The conditions of plants include plant diseases which refer to any persistent irritation resulting in plant damage and characteristic symptoms resulting from algae or fungi infection or from environment surrounding the plant (e.g., soil-related plant diseases). The plant diseases include, for example, fungal infection occurring to the leaves of the plants (foliar diseases), the flowers of the plants (flower diseases), the fruits or vegetables during or after harvesting (post-harvest diseases).

The foliar diseases mentioned herein include, for example, Botrytis, Collectotrichum, Mycosphaella, Septoria, Fusarium, Powdery Mildew and Downy Mildew. The flower diseases mentioned herein include, for example, Botrytis Blight. The target post-harvest diseases of fruits and vegetables include, for example, Penicillium, Geotrichum, and Botrytis. The composition of the present invention comprising the ATCC PTA-7736 strain (or the strain in combination with other carriers) can be sprayed to a plant (e.g., leaves fruits, flowers, vegetables, branches, and trunks). The composition can also be placed in trays in or on beehive entrances. The bees will be coated with the composition by electrostatic action and the composition will be taken to the flowers to control, prevent or treat the fungal infection in flowers.

In another embodiment of the present invention, a life form is a human or an animal. The conditions of human and animals include diseases caused by or related to the presence of fungi and/or algae (human diseases and animal diseases). The said diseases include, but are not limited to allergy to the fungi articles, fungal infections of respiration system, genitourinary system, bone, eye, nail, hair and skin. The composition comprising the ATCC PTA-7736 strain (or the strain with a pharmaceutically acceptable carrier) can be applied to the air human and animals through, for example, spray, oral, parenteral, mucosal, transmucosal, inhale, nasal, virginal, rectal, topical, or transdermal drug delivery routes.

In another embodiment of the present invention, a sample is a tangible article. Examples of tangible articles include, for example, residential and commercial buildings, constructions, boat, toys, vehicles, furniture, art, book, and food. A condition of a tangible article refers to a condition that is caused by or related to the presence or overgrowth of algae and/or fungi on, in, surrounding the tangible article. The composition of the present invention comprising the ATCC PTA-7736 strain (or the strain with a carrier) can be applied to the air surrounding the tangible article or directly in/on the tangible article to prevent or treat the said condition.

In one embodiment of the invention, the composition comprises a hydrophobic carrier and the microbial agent to effectively distribute the microbial agent to a sample (hydrophobic composition). A preferred hydrophobic carrier is selected from a group comprising carbon minerals, leonardite, plastic, wood powder, plant materials, clay and combinations thereof. The carbon minerals are preferably selected from a group comprising coal, charcoal, graphite, carbon black, oil, straw and combinations thereof. The hydrophobic carrier preferably comprises activated carbon or charcoal specifically designed for distribution onto and into water. The carbon formulations also confer UV protection to the microbial agent during exposure to sunlight and act as an inoculum source for the microbial agent as it passes into the liquid medium.

Carbon in various forms has been used in commercial water treatments previously, but only as a filter or absorbent. The hydrophobic properties of the hydrophobic composition allow the composition to resist mixing with the water and instead form a thin film that rapidly disperses over the water surface resulting in its effective application to the water surface. Accordingly, in addition to the preferred hydrophobic carriers, one skilled in the art will appreciate that any hydrophobic or carbon composition may be employed as a hydrophobic carrier.

In a preferred embodiment of the invention, the microbial agent is formulated onto a hydrophobic carrier to produce a material with about 4.5×10⁶ cfu/g of hydrophobic composition to about 4.5×10¹⁰ cfu/g of hydrophobic composition. In another aspect of the invention, the hydrophobic composition may also include a mineral fertilizer. The mineral fertilizer allows the hydrophobic composition to be applied to water films on the surface of plants, such as commercial turf. The hydrophobic composition may be added to a mineral fertilizer for application to commercial turf and similar applications. In this aspect of the invention, the hydrophobic composition may also be applied to the surface of water in the form of a water-soluble bag for use as an algaecide for blue-green algae (cyanobacterium), microscopic algae, filamentous algae, string algae, hair algae, and blanket weed.

Another aspect of the present invention is a method of delivering microbes to a water application uniformly and efficiently comprising, the step of applying a hydrophobic composition to a water application wherein the hydrophobic composition comprises a hydrophobic carrier and a microbial agent. In addition to forming a thin uniformly distributed film over the water surface, the microbial agents may be released into the body of water and move downward through the primary regions where the target pests or diseases are located during and after surface distribution. The microbial agent then interacts with the target pests or diseases and the hydrophobic carrier serves as a reservoir for the microbial agent. In the case of bacteria, the hydrophobic carrier provides a matrix for further growth.

Another aspect of the invention is a method for improving the water quality of water. In a preferred embodiment of the invention a hydrophobic composition is introduced to water. The hydrophobic composition disperses rapidly as a thin film over the surface of the water and downward through primary regions of the water. The microbial agents of the hydrophobic composition are released into the water application, thereby treating and improving the water quality of the water application.

In further aspects of the invention, the hydrophobic compositions treat and improve water quality by controlling algae growth, de-odorizing the water application, suppressing water eutrophication, and reducing total suspended solids, ammonia, free nitrates and phosphates in water. The hydrophobic compositions of this aspect of the invention are delivered at rates ranging from about 0.1 g/m² to about 1000 g/m², most preferably from about 1 g/m² to about 1.5 g/m². The hydrophobic composition may also be delivered at rates ranging from about 100 cfu/m² to about 1×10¹¹ cfu/m².

In a preferred embodiment of the invention, a hydrophobic composition comprising the microbial agent is formulated with charcoal to produce a hydrophobic composition with about 4.5×10⁶ cfu/g of hydrophobic composition to about 4.5×10¹⁰ cfu/g of hydrophobic composition. The composition may be applied to water applications to control algae growth, de-odorize the water application, suppress water eutrophication, and reduce total suspended solids, ammonia, free nitrates and phosphates in the water applications whereby the hydrophobic composition is efficiently and uniformly dispersed over the surface of and into the water application.

In a preferred embodiment, the microbial agent is used directly or with a carrier (e.g., hydrophobic carrier) to control algae growth and improve water quality in water applications. Another embodiment of the invention is a method for treating water and controlling algae growth in water applications comprising the step of applying a composition comprising the microbial agent (or the agent with a carrier) to a sample. In another embodiment of the invention, the hydrophobic composition may be supplemented with ground cotton stubble.

### EXAMPLE 1

### Effect of Hydrophobic Composition in an Algae Infested Water Application

Various strains of Bacillus subtilis were formulated with charcoal to produce a hydrophobic composition with 4.5×10⁸ cfu/g of hydrophobic composition. The hydrophobic composition was then applied to the surface of an algae infested pond in Riverside, Calif., USA at a range of rates from 0.1-1.5 g/m². The water quality of the pond was monitored over a ten day period. On certain days during the ten day period, the algae in the pond was rated from 0 to 5, with 0 indicating the worst water quality and a high incidence of algae and 5 indicating good water quality and no incidence of algae.

As the rates of application of the formulation increased, the water quality increased. In addition, generally, as the rates of formulation increased, the water quality increased at faster rates. Table 1 illustrates the results of the study.

TABLE 1

| Rate of | | | | | |
|---|---|---|---|---|---|
| Formulation | Day 0 | Day 3 | Day 5 | Day 7 | Day 10 |
| 0 g | 2 | 2 | 2 | 2 | 2 |
| 0.1 g | 2 | 2 | 2 | 2 | 2 |
| 0.5 g | 2 | 2 | 3 | 3 | 3 |
| 1 g | 2 | 2 | 2 | 3 | 4 |
| 1.5 g | 2 | 2 | 4 | 4 | 5 |

0 = Worst Water Quality, high incidence of algae
5 = Water Quality good, no incidence of algae.

### EXAMPLE 2

### Suppression of Algae in Hydroponic Systems

Example 2 illustrates the suppression of algae in hydroponic systems. The hydrophobic composition of Example 1 was supplemented with ground cotton stubble to create Example 2. The composition was then applied to the drainage canals of a seven acre hydroponics facility in Florence, Oreg., USA at a rate of 1.5 g/m². The water quality of the hydroponics facility increased at a dramatic rate after introduction of the hydrophobic composition. The results of the study are illustrated in Table 2.

TABLE 2

| Treatment Rate | Day 0 | Day 3 | Day 7 | Day 10 |
|---|---|---|---|---|
| 1.5 g/m² | 1 | 2.5 | 4 | 4.5 |

0 = Worst Water Quality, high incidence of algae
5 = Water Quality good, no incidence of algae.

### EXAMPLE 3

### Hydrophobic Composition in a Water Application

Example 3 illustrates the uniform distribution of the hydrophobic composition in a water application. A water column was set up in Reedley, Calif., USA. Various strains of Bacillus subtilis not including the ATCC PTA-7736 strain were formulated with charcoal to produce a hydrophobic composition with 4.5×10⁸ cfu/g of hydrophobic composition. The hydrophobic composition was then applied to the center of the surface of water as a small pile, at a range of rates from 0.1-1.5 g/m². The rate at which the material spread across the water surface and through the column is described in Table 3. As Table 3 illustrates, the hydrophobic composition disperses rapidly over a water surface and downwards through the water column.

TABLE 3
Time to percentage coverage

| | 25% | 50% | 75% | 100% |
|---|---|---|---|---|
| Water Surface | 0.4 min | 1.25 min | 2.3 min | 3.5 min |
| Water Column | 4.75 min | 7.5 min | 9.5 min | 12.3 min |

### EXAMPLE 4

### Hydrophobic Compositions on Commercial Turf

Example 4 illustrates the effect of hydrophobic compositions on commercial turf. The hydrophobic composition of Example 1 was applied to commercial turf at Morro Bay, Calif., USA at rates of 2 g/m² and 4 g/m². The alga incidence and the incidence of certain foliage diseases were measured 14 days later. The turf health was also measured, with 0 indicating healthy turf and 5 indicating unhealthy turf. The results of this study are illustrated in Table 4. As the rates of application of the formulation increased, the incidence of algae and leaf disease decreased, while the health of the turf increased.

TABLE 4

| | Algae | Leaf Disease | Turf |
|---|---|---|---|
| Rates | Incidence | Incidence* | Health** |
| Control | 15% increase | 22% increase | 2.3 |
| 2 g/m² | −33% | −55% | 1.5 |
| 4 g/m² | −65% | −75% | 1 |

*Main disease of turf at this site was leaf anthracnose caused by Collectotrichum.
**Turf health Index 0 = no necrotic patches, growth excellent, color excellent.
5 = Dead

### EXAMPLE 5

### Hydrophobic Compositions in Aquariums and Fish Tanks

Example 5 illustrates the use of hydrophobic compositions in aquariums and fish tanks. Algae growth in aquariums and fish tanks is a common problem. The hydrophobic compositions of this invention may be applied to aquariums and fish tanks to treat the water and thus control the algae growth.

The hydrophobic compositions of the invention may be placed on the water surface of an aquarium or fish tank. The hydrophobic composition rapidly disperses as a thin film over the water surface, and then this film enters the liquid phase as a suspension. The Bacillus subtilis strain ATCC PTA-7736 are released into the body of water during dispersal over the surface of water and as the composition moves downwards through the water column to treat the algae growth in the aquarium or fish tank.

During treatment of the aquarium or fish tank for algae growth, the fish and any other aquatic/marine life forms do not need to be removed from the aquarium or fish tank. The microbial agents and hydrophobic carriers of the hydrophobic composition are not harmful to aquatic/marine life or wildlife, unlike other methods that employ chemicals or pesticides. The methods of this invention that employ microbial agents are environmentally safe.

### EXAMPLE 6

### Hydrophobic Compositions in Feeding Troughs for Animals

Example 6 illustrates the use of hydrophobic compositions in feeding troughs for animals. The hydrophobic composition of Example 1 may be introduced into the feeding troughs of animals. The hydrophobic composition controls the growth of target pests and diseases. Further, the hydrophobic composition is not harmful to the animals. As a result, the frequency with which the feeding troughs need to be cleaned and replaced may be decreased.

### EXAMPLE 7

### Laboratory Studies with Bacillus subtilis Strain ATCC PTA-7736 on Algae

Micro-algae are collected from natural sources (ponds and streams) and comprise mainly unicellular algae, blue green and filamentous algae. 20 jars are prepared to contain 200 mL tap water and 0.01 g Osmacote. Each jar is inoculated with 1 ml aliquot of the algae sample. All jars are placed on a shaker and illuminated for 12 h daily. After 3 days, the following treatments are administered: 1. No treatment; 2.5×10³ cfu/g; 3.5×10⁵ cfu/g; 4.5×10⁷ cfu/g; and 5.5×10⁹ cfu/g.

Each treatment is applied to 4 replicate jars. The jars are randomized and incubated. All treatments are inspected daily as a 3 mL sample is taken from each jar to measure its optical transmitted absorbance at 550 nm. The measurements are repeated twice. The results are averaged and presented below.

| Treatment | Day 0 | Day 2 | Day 3 | Day 4 | Day 5 | Day 6 |
|---|---|---|---|---|---|---|
| 1 | 0.77a | 0.78a | 0.82a | 0.76a | 0.84a | 0.85a |
| 2 | 0.86a | 0.83a | 0.74a | 0.77a | 0.54a | 0.42b |
| 3 | 0.76a | 0.55ab | 0.32b | 0.15b | 0.16b | 0.12c |
| 4 | 0.87a | 0.23b | 0.11b | 0.15b | 0.12b | 0.14c |
| 5 | 0.78a | 0.24b | 0.16b | 0.13b | 0.11b | 0.12c |

a-d are statistical separation groups according to a Student's T-test at a probability level of P = 0.05.

a-d are statistical separation groups according to a Student's T-test at a probability level of P=0.05.

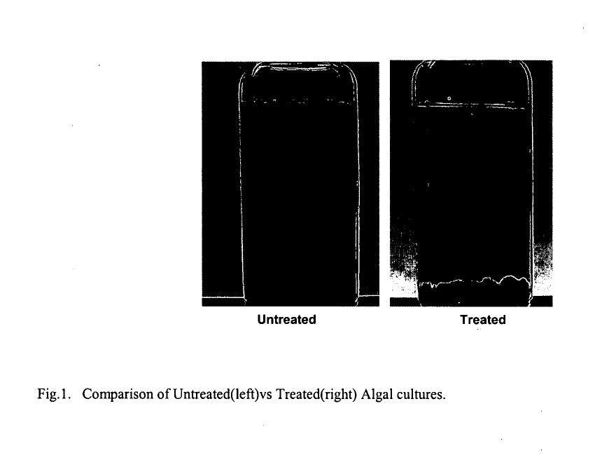

The comparison of Bacillus subtilis ATCC PTA-7736 treated and untreated Algal cultures are shown in FIG. 1.

This example illustrates that the Bacillus subtilis strain ATCC PTA-7736 suppresses Microscopic and Filamentous algal growth.

### EXAMPLE 8

### Field Trials with Bacillus subtilis Strain ATCC PTA-7736

The Bacillus subtilis strain ATCC PTA-7736 is propagated by semi solid fermentation technique as following:

Soybean meal is mixed with water at a 70 to 30 weight ratio. The resulting media is steam sterilized and cooled to 38 degree centigrade, and the Bacillus subtilis strain ATCC PTA-7736 is inoculated. The fermentation process lasts about 24 h at temperature between 25-40° C. The media is turned twice during the above process. Upon completion of the fermentation process, the soybean meal is dehydrated with hot air and then pulverized into fine powder.

Activated charcoal is blended with the fine powder obtained above at a 50:50 weight ratio. The resulting material is then applied into a small lake on a golf course in Colton, Calif. The lake had visual evidence of filamentous algae and it was also apparent that there was a stagnant odor to the pond. The lake size is approximately 1500 m² and with an approximate depth of 1.3 m. 20×1 kg of the B.subtilis strain was applied to the lake's water surface form from equidistant points around the shoreline. The lake was monitored over 10 days for evidence of algal suppression.

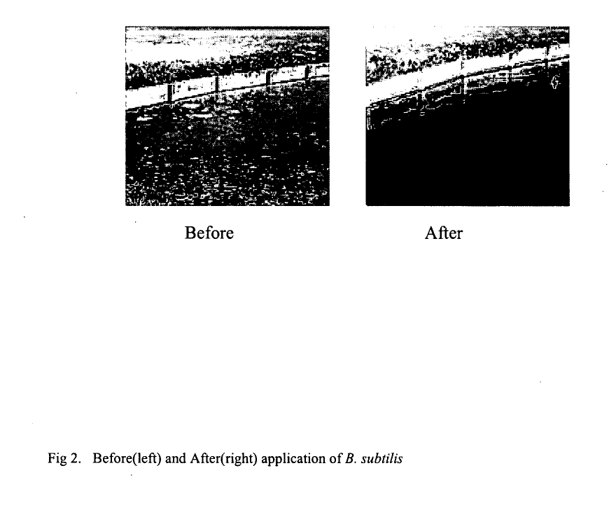

Results are shown in FIG. 2: After 10 days it was apparent that there had been a visual improvement to the lakes water quality. Greenex (the Bacillus subtilis strain ATCC PTA-7736 ) eradicated Filimentous and microscopic algae from this site. Upon microscopic examination of the algae, the bacteria were seen to adhere to the alga surface and digest the surface tissues and gels ultimately resulting in cell lysis.

### EXAMPLE 9

### Laboratory Studies with Bacillus subtilis Strain ATCC PTA-7736 on Fungus

A plant pathogenic fungus, Fusarium oxysponim was inoculated on Potato Dextrose Agar (PDA) in two Petri dishes that were prepared using conventional method. The Bacillus subtilis strain ATCC PTA-7736 was applied in the shape of a cross in the middle of one Petri dish. The strain ATCC PTA-7736 was applied on another Petri dish as two parallel lines. The two Petri dishes were incubated for two days.

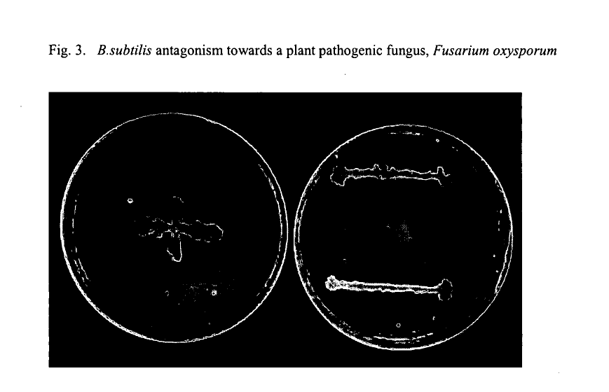

Result is shown in FIG. 3: The presence of the Bacillus subtilis strain ATCC PTA-7736 is antagonist toward the growth of Fusarium oxysporum.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A method of preventing or treating a condition of a sample comprising a step of applying a composition to the sample, wherein the composition comprising an isolated Bacillus subtilis strain ATCC PTA-7736.

2. The method of claim 1 wherein the composition further comprises a carrier selected from the group consisting of a liquid carrier, a solid carrier, a hydrophilic carrier, a hydrophobic carrier, and a pharmaceutically acceptable carrier.

3. The method claim 2 wherein the hydrophobic carrier is selected from the group consisting of carbon minerals, plastics, wood powder, plant materials, clays or combinations thereof.

4. The method of claim 3 wherein the carbon minerals are selected from a group consisting of coal, charcoal, activated charcoal, graphite, carbon black, oil, straw or combinations thereof.

5. The method of claim 1 wherein the condition is the presence or overgrowth of unwanted organisms in, on, or surrounding a sample and the results thereof, wherein the preventing or treating the condition becomes desirable, and wherein the unwanted organisms include algae or fungi.

6. The method of claim 1 wherein the sample is a subject which has propensity to allow the presence or overgrowth of unwanted organism in, on or surrounding the subject, wherein the unwanted organisms are algae or fungi.

7. The method of claim 6 wherein the subject is liquid, gas, solid matter, a life form, or a tangible article.

8. The method of claim 7 wherein the liquid is water.

9. The method of claim 8 wherein the water in a lake, a river, a canal, a pond, a tank, or a reservoir.

10. The method of claim 7 wherein the gas is air.

11. The method of claim 10 wherein the air is the atmosphere, the processed air, or the air confined in a close container or space (e.g., the air in a building or a car).

12. The method of claim 7 wherein the solid matter is soil.

13. The method claim 7 wherein a life form is a plant, a human or an animal.

14. The method of claim 12 wherein a plant includes a tree, a flower, a leaf, a trunk, a branch, a fruit, a root, a vegetable, a herb, a fern, and a moss.

15. The method of claim 14 wherein the condition of a plant is a soil-related plant disease, a foliar disease, a flower disease, or a post-harvest disease.

16. The method of claim 15 wherein the soil-related plant disease is Phytophthora, Pythium, Verticillium, Rhizoctonia, or Fusarium.

17. The method of claim 15 wherein the foliar disease is Botrytis, Collectotrichum, Mycosphaella, Septoria, Fusarium, Powdery Mildew, or Downy Mildew.

18. The method of claim 15 wherein the flower disease is Botrytis Blight.

19. The method of claim 15 wherein the post-harvest diseases is Penicillium, Geotdchum, or Botrytis.

20. The method of claim 13 wherein the condition of a human or an animal includes an allergy to the fungi articles or fungal infections of respiration system, genitourinary system, bone, eye, nail, hair or skin.

21. The method of claim 20 wherein the condition is treated or prevented by applying the composition to the human or the animal through spray, oral, parenteral, mucosal, transmucosal, inhale, nasal, virginal, rectal, topical, or transdermal drug delivery routes.

22. The method of claim 7 wherein the tangible article is a residential building, a commercial building, a construction, a boat, a toy, a vehicle, a piece of furniture, a piece of art, a book, and food.

23. The method of claim 1 wherein the composition can be applied to a sample by spray, addition, or mixing.

24. The method of claim 8, wherein the composition is applied to the water application at rates of about 0.1 g/m² to about 1000 g/m².

25. The method of claim 8, wherein the composition is applied to the water at rates of about 100 cfu/m² to about 1×10¹¹ cfu/m².

26. The method of claim 1 wherein the strain is combined with other organisms selected from the group consisting of amoebas, fungi, bacteria, nematodes and protozoans and combinations thereof.

27. An isolated, biologically pure Bacillus subtilis strain, which is deposited under American Type Culture Collection with a patent deposit designation number PTA-7736 (ATCC PTA-7736).
